(19) United States
(12) Patent Application Publication
(10) Pub. No.: US 2019/0062322 A1
(43) Pub. Date: Feb. 28, 2019

(54) PUMA INHIBITOR, METHOD FOR PREPARATION THEREOF, AND USE THEREOF

(71) Applicant: FOURTH MILITARY MEDICAL UNIVERSITY, Xi'an City (CN)

(72) Inventors: Hui CHEN, Xi'an City (CN); Jiyuan LIU, Xi'an City (CN); Nan ZHOU, Xi'an City (CN); Shengyong ZHANG, Xi'an City (CN)

(73) Assignee: FOURTH MILITARY MEDICAL UNIVERSITY, Xi'an City (CN)

(21) Appl. No.: 16/087,413

(22) PCT Filed: Sep. 21, 2016
(86) PCT No.: PCT/CN2016/099536

§ 371 Date: Sep. 21, 2018

(30) Foreign Application Priority Data
Apr. 19, 2016 (CN) 201610242882.1

(51) Int. Cl. C07D 417/04; A61P 9/04; A61P 9/10; A61P 25/28; A61P 31/18; C07D 417/14

(52) U.S. Cl. CPC C07D 417/04; A61P 9/04; C07D 417/14; A61P 25/28; A61P 31/18; A61P 9/10

(57) ABSTRACT

The present invention relates to the technical field of medicine, and specifically relates to a PUMA inhibitor, a method for preparation thereof, and a use thereof. The PUMA inhibitor is the compound as shown in (I) or a pharmaceutically acceptable salt thereof. The definitions of its substituent groups are as described in the description. The compound (I) or pharmaceutically acceptable salt thereof is capable of targeting a PUMA protein and has very good physicochemical properties, very good apoptosis resistance and radiation protection effects; it can selectively inhibit the competitive combined effect of a PUMA protein and a Bcl-2 apoptosis-resistant protein, block apoptosis, and effectively protects against bone marrow damage. The present invention provides a new idea for, and powerful proof of, the application of a PUMA-protein small molecule inhibitor in radiation-protection pharmaceuticals, and is likely to become a highly effective, low-toxicity, and stable clinical radiation-protection pharmaceutical.

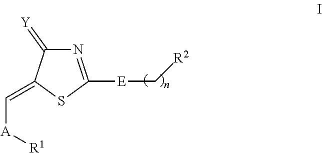

### FIELD OF THE INVENTION

The invention relates to the technical field of medicine and provides a class of new inhibitors targeting PUMA protein, method of their preparation and use as medications.

### BACKGROUND OF THE INVENTION

Ionizing radiation was developed rapidly in medical therapy, nuclear electricity generation, radiation breeding, terror attacks and nuclear weapons. Uncontrolled exposure to high-dose radiation presents the greatest threat and challenge to the civilized world. In these cases, radiation causes damage to normal cells and, hence, can result in injury to the human body. Depending upon the duration of exposure, the sensitivity of the organs, and the dose received, radiation exposure could lead to a myriad of deleterious effects.

Total body-irradiation will result in hematopoietic damage with possible lethality as the consequence of induced apoptosis in cells. Current radiation treatment approaches are primarily exploiting regulation of hematopoietic system. Thus, radioprotectant treatment with targeted reagents have been shown to increase recovery of irradiated body. Understanding the molecular mechanisms of radiation-induced cell apoptosis provided potential targets for developing an effective treatment to ameliorate radiation-induced injury.

Bcl-2 protein family, the first identified apoptotic regulator, which regulates mitochondrial apoptosis, is composed of both proapoptotic and antiapoptotic members that cooperate through protein-protein interactions (PPI) to mediate the apoptotic pathway.

PUMA (a BH3-only proapoptotic protein) is a member of Bcl-2 protein family and it is critical for inhibition of anti-apoptotic proteins and initiation of apoptosis signaling. PUMA plays an essential role in apoptosis induced by a variety of stimuli, such as radiation. Its absence contributes to the failure of apoptosis induced by p53. The overexpressed PUMA directly targets all 5 known anti-apoptotic Bcl-2 family members (Bcl-2, Bcl-x1, Mcl, A1, Bcl-x) with high affinities by its BH3 domain, consequently triggering apoptosis by inducing mitochondrial dysfunction and caspase activation. Moreover, according to the protein crystal structure, the key binding site of PUMA contains 9 base pairs. So, PUMA is considered as a potential target for developing an effective treatment to protect those cells exposed to radiation.

So, inhibition of PUMA provides a profound benefit for the long-term survival after irradiation. PUMA may be a potential target for developing an effective treatment aimed to protect cells from lethal radiation. This invention relates to new inhibitors targeting PUMA protein. In particular, the invention relates to effective radioprotectants with low-toxicity and high-stability.

### DETAILED DESCRIPTION OF THE INVENTION

This invention relates to new inhibitors targeting PUMA protein. These targeted inhibitors will be administrated to radiation-induced damage. In addition, they may be administrated to apoptosis-related disease, such as myocardial ischemia, ischemia-reperfusion injury, cardiac failure, neurodegenerative disorders such as Alzheimer's disease and virus infection such as AIDS virus infection as well.

In one aspect, compounds of formula I or their pharmaceutically acceptable salts are provided.

In another aspect, a method of synthesizing compounds of formula I or their pharmaceutically acceptable salts is provided.

In a further aspect, the invention provides use of compounds of formula I or their pharmaceutically acceptable salts in the preparation of a medicament.

The invention provides compounds of formula I or their pharmaceutically acceptable salts.

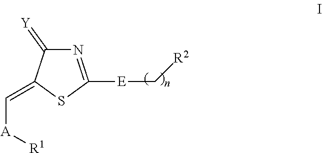

Wherein:

A is C₅˜C₁₀ aryl, or 5˜10 membered heteroaryl containing 1˜3 atom(s) selected from oxygen, nitrogen and sulfur;

Specifically, A is:

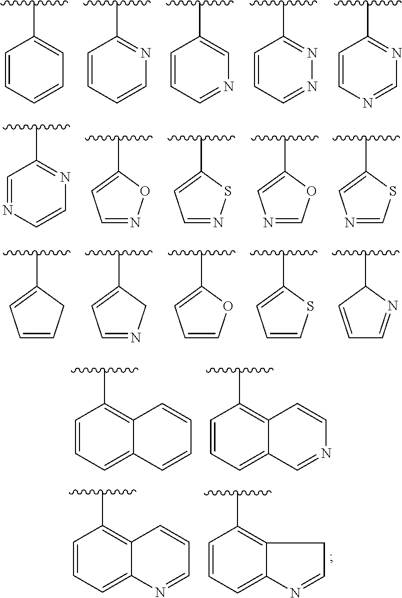

Preferably, A is C₅˜C₆ aryl, or 5˜6 membered heteroaryl containing 1˜2 heteroatom(s) selected from oxygen, nitrogen and sulfur;

More preferably, A is benzyl or pyridazinyl.

Group A will interact with key amino acids of PUMA by hydrophobic bond when binding happens.

E is 5˜6 membered saturated or unsaturated aliphatic ring containing 0˜2 heteroatom(s) selected from oxygen, nitrogen and sulfur, or C₃˜C₆ alkyl, —NH—C₁˜C₃ alkyl, —O—C₁˜C₃ alkyl, —C₁˜C₃ alkyl-NH—C₁˜C₃ alkyl-, —C₁˜C₃ alkyl-O—C₁˜C₃ alkyl, —NH—C₁˜C₃ alkyl-NH—, —O—C₁˜C₃ alkyl-O—;

Specifically, E is:

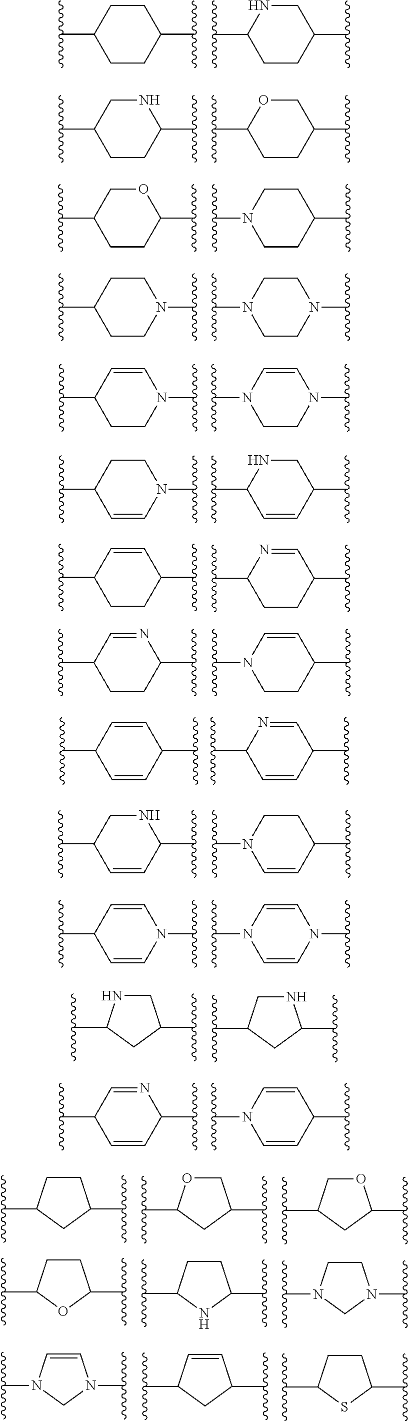

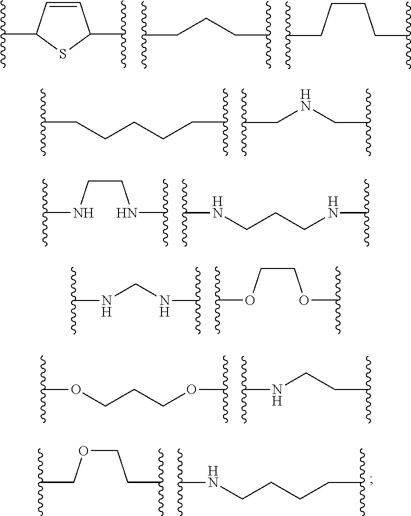

Preferably, E is 5˜6 membered saturated aliphatic ring containing 0˜2 heteroatom(s) selected from oxygen, nitrogen and sulfur;

More preferably, E is piperazinyl or piperidyl.

Group E will interact with key amino acids of PUMA by hydrogen bond when binding happens.

Y is O, S, NH or CH₂, preferably, Y is O.

R¹ is H, C₁˜C₆ alkyl, C₁˜C₆ alkoxy, halogen (F, Cl, Br, I), —OH, —NH₂, or 1˜3 R³-substituted C₅˜C₆ aryl, 5˜6-membered heteroaryl containing 1˜2 heteroatom(s) selected from oxygen and nitrogen, R³ is H, C₁˜C₆ alkyl, C₁˜C₆ alkoxy, halogen (F, Cl, Br, I), —OH, —NH₂;

Specifically, R¹ is H, C₁˜C₆ alkyl, C₁˜C₆ alkoxy, halogen (F, Cl, Br, I), —OH, —NH₂,

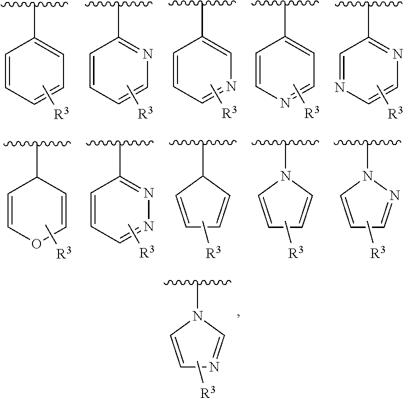

R² is H, —OH, —NH₂, —SH, —COOH, —CONH₂, —SO₂NH₂, —SO₃H, —CH(OH)—CH₂—OH.

Group R² will interact with key amino acids of PUMA as hydrogen bond donor when binding happens.

n is integer from 1 to 5, specifically, n is 1, 2, 3, 4, 5.

The term “aryl” means a carbocyclic aromatic system not containing heteroatom.

The term “heteroaryl” means heterocyclic aromatic ring system containing heteroatom.

The term “alky” means linear or branched alkyl.

In above description, A, E, Y, R¹, R² and n can be any combination of A, E, Y, R¹, R² within the limitation of common, specifically, preferably, more preferably.

Preferably, compounds of formula I or their pharmaceutically acceptable salts are compounds of formula II and their pharmaceutically acceptable salts:

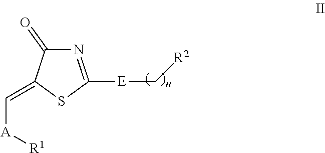

Wherein, A, E, R¹, R², and n are as defined above.

In one embodiment, compounds of formula I or II or their pharmaceutically acceptable salts are compounds 1˜12 and their pharmaceutically acceptable salts:

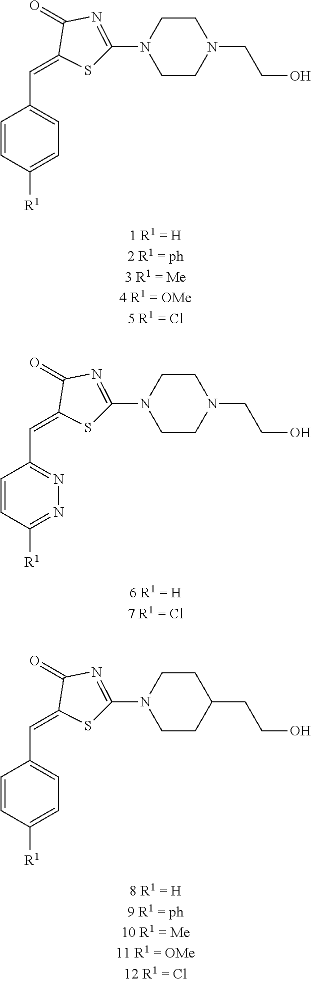

In another embodiment, compounds of formula I or II or their pharmaceutically acceptable salts are compounds as follows and their pharmaceutically acceptable salts.

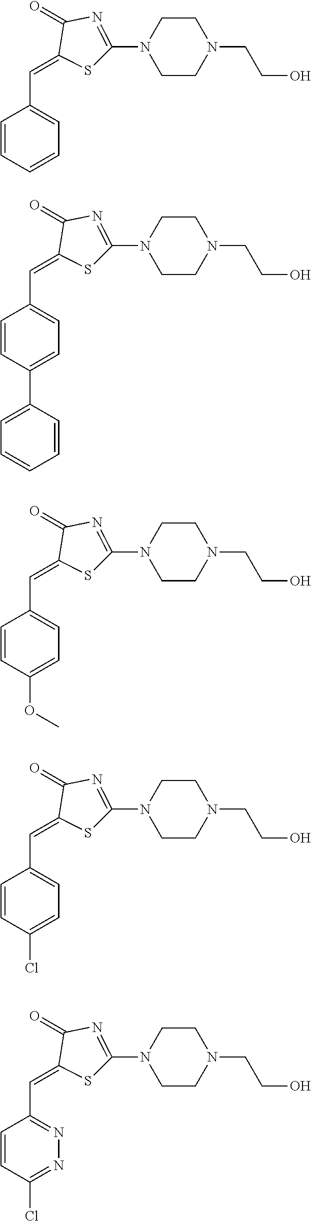

The present invention provides the preparation method of compounds of formula I or their pharmaceutically acceptable salts. The general procedure is as follows:

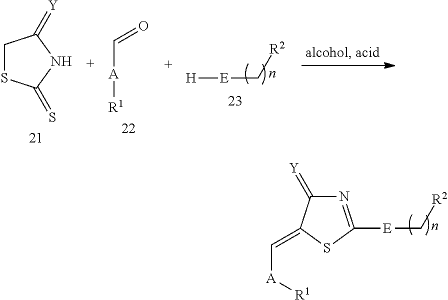

Adding compounds 22 and 23 into the mixture of compound 21, alcohol (eg. methanol) and acid (eg. glacial acetic acid) under N₂ atmosphere. The resulting mixture was further heated to reflux. When reaction was completed, cooling the obtained solution; filtering off the precipitated crystalline solid, then washing and purifying by silica gel column chromatography.

Wherein, A, E, R¹, R², R³ and n are as defined above.

In one aspect, the use of compounds of formula I or their pharmaceutically acceptable salts in the preparation of PUMA inhibitors are provided.

In another aspect, the use of compounds of formula I or their pharmaceutically acceptable salts in the preparation of radioprotectant and antiapoptotic drugs are provided.

In a further aspect, the invention provides the use of compounds of formula I or their pharmaceutically acceptable salts in the preparation of medicament for treating radiation-induced hematopoietic damage, myocardial ischemia, Ischemia-reperfusion injury, cardiac failure, Alzheimer's disease and AIDS virus infection.

In a further aspect, the invention provides the use of compounds of formula I or their pharmaceutically acceptable salts in the preparation of antiapoptotic drugs.

Moreover, the invention provides a pharmaceutical composition, comprising compounds of formula I or their pharmaceutically acceptable salts and pharmaceutically acceptable excipients.

The compounds of formula I or their pharmaceutically acceptable salts are PUMA-targeted and have good physicochemical properties, antiapoptotic activities and radioprotection effect. They can interrupt the binding between PUMA and anti-apoptotic Bcl-2 protein selectively, and block apoptosis, so to protect against the radiation-induced hematopoietic damage. So, this invention provides the inhibitors of PUMA, which are a profound benefit for the long-term survival after irradiation and then to be novel radioprotectants with highly effective, low-toxicity and high-stability in clinical application.

### EXAMPLES

The invention will be described in greater detailed in conjunction with the following specific examples, but the invention is not limited to this. The following procedure is conventional approach, and the involved materials are commercially available.

### Preparation Example 1

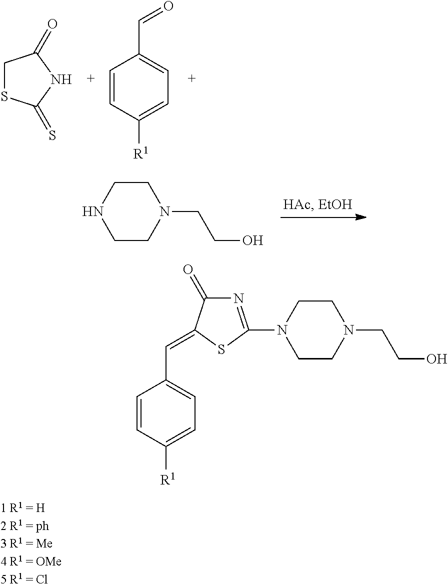

To a suspension of rhodanine (2.0 g, 15 mmol) in dry methanol (20 mL) and glacial acetic acid (0.1 mL), N-(2-Hydroxyethyl)piperazine (2.9 g, 22.6 mmol) and benzaldehyde (22.6 mmol) under N₂, and the resulting mixture was further stirred under reflux for 1-2 h. Reaction was completed (checked by TLC analysis) and the obtained yellow solution was cooled in an ice bath; the precipitated crystalline solid was filtered off, washed with cooled methanol, and dried under vacuum to obtain the crude product. The crude product was subjected to silica gel column chromatography using dichloromethane/methanol (10:1) as mobile phase to afford the desired compound 1, yield is 35-55%.

Yellow solid, Yield=46%. ¹H NMR (400 MHz, CDCl₃) δ=7.83 (s, 1H), δ=7.56 (d, 2H), δ=7.47 (m, 2H), δ=7.41 (t, 1H), δ=4.1 (t, 2H), δ=3.69 (m, 4H), δ=2.72 (t, 2H), δ=2.66 (m, 4H). ¹³CNMR (400 MHz, CDCl₃) 180.84, 175.22, 134.16, 131.94, 129.78, 129.74, 129.03, 127.88, 59.26, 58.05, 52.53, 52.11, 48.68, 48.34, HRMS(ESI) calcd. for C₁₆H₁₉N₃O₂S: 317.1 [M+H]⁺, found 317.4 [M+H]⁺.

### Preparation Example 2

Compound 2 was prepared with a method similar to preparation example 1, wherein benzaldehyde was replaced by 4-Biphenylcarboxaldehyde in this procedure.

Yellow solid, Yield=52.9%. ¹H NMR (400 MHz, CDCl₃) δ=7.87 (s, 1H), δ=7.69 (d, 2H), δ=7.63 (m, 4H), δ=7.48 (t, 2H), δ=7.42 (t, 1H), δ=5.32 (s, 1H), δ=4.1 (t, 2H), δ=3.71 (m, 4H), δ=2.73 (t, 2H), δ=2.68 (m, 4H) HRMS(ESI) calcd. for C₂₃H₂₇N₃O₂S, 409.2 [M+H]⁺, found 409.5 [M+H]⁺.

### Preparation Example 3

Compound 3 was prepared with a method similar to preparation example 1, wherein benzaldehyde was replaced by p-methyl benzaldehyde in this procedure.

Yellow solid, Yield=49.1%. ¹H NMR (400 MHz, CDCl₃) δ=7.81 (s, 1H), δ=7.43 (d, 2H), δ=7.27 (t, 3H), δ=4.12 (t, 2H), δ=3.70 (m, 4H), δ=2.73 (t, 2H), δ=2.68 (m, 4H), δ=2.40 (s, 3H) HRMS(ESI) calcd. for C₁₇H₂₁N₃O₂S, 331.4 [M+H]⁺, found 331.8 [M+H]⁺.

### Preparation Example 4

Compound 4 was prepared with a method similar to preparation example 1, wherein benzaldehyde was replaced by 4-methoxybenzaldehyde in this procedure.

Yellow solid, Yield=47.6%, ¹H NMR (400 MHz, CDCl₃) δ=7.78 (s, 1H), δ=7.49 (d, 2H), δ=6.98 (d, 2H), δ=4.10 (t, 2H), δ=3.86 (s, 3H), δ=3.69 (m, 4H), δ=2.71 (t, 2H), δ=2.69 (m, 4H) HRMS(ESI) calcd. for C₁₇H₂₁N₃O₃S, 347.4 [M+H]⁺, found 347.7 [M+H]⁺.

### Preparation Example 5

Compound 5 was prepared with a method similar to preparation example 1, wherein benzaldehyde was replaced by 4-Chlorobenzaldehyde in this procedure.

Pale green solid, Yield=37.2%, ¹H NMR (400 MHz, CDCl₃) δ=7.75 (s, 1H), δ=7.43 (dd, 4H), δ=4.11 (t, 2H), δ=3.69 (m, 4H), δ=2.72 (t, 2H), δ=2.67 (m, 4H) HRMS(ESI) calcd. for C₁₆H₁₈ClN₃O₂S 351.8 [M+H]⁺, found 360.0 [M+H]⁺.

### Preparation Example 6

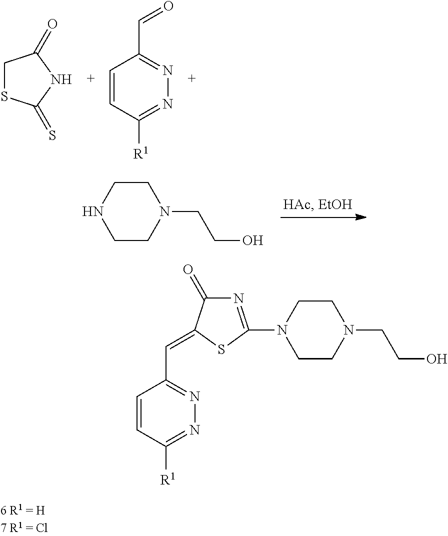

To a suspension of rhodanine (2.0 g, 15 mmol) in dry methanol (20 mL) and glacial acetic acid (0.1 mL), N-(2-Hydroxyethyl) piperazine (2.9 g, 22.6 mmol) and pyridazine-3-carbaldehyde (22.6 mmol) under N₂, and the resulting mixture was further stirred under reflux for 4 h. Reaction was completed (checked by TLC analysis) and the obtained solution was cooled in an ice bath; the precipitated crystalline solid was filtered off, washed with cooled ethanol, and dried under vacuum to obtain the crude product. The crude product was subjected to silica gel column chromatography using dichloromethane/methanol (10:2) as mobile phase to afford the desired compound 6.

Yellow solid, Yield=37.1%. ¹H NMR (400 MHz, CDCl₃) δ=8.89 (d, 1H), δ=7.55 (d, 1H), δ=7.44 (d, 1H), δ=7.39 (s, 1H), δ=4.20 (br, 11H), δ=4.04 (t, 2H), δ=3.61 (m, 4H), δ=3.12 (t, 2H), δ=2.56 (m, 4H) ¹³C NMR (400 MHz, CDCl₃) 170.28, 169.24, 154.79, 134.96, 132.94, 130.27, 128.31, 129.03, 58.94, 57.93, 54.72, 52.48, 48.77, 48.39, HRMS(ESI) calcd. for C₁₅H₁₈N₄O₂S: 318.1 [M+H]⁺, found 318.8 [M+H]⁺.

### Preparation Example 7

Compound 7 was prepared with a method similar to preparation example 6, wherein pyridazine-3-carbaldehyde was replaced by 6-Chloro-pyridazine-3-carbaldehyde in this procedure.

Yellow solid, Yield=22.9%. ¹H NMR (400 MHz, CDCl₃) δ=7.58 (d, 1H), δ=7.35 (d, 1H), δ=7.31 (s, 1H), δ=4.62 (br, 1H), δ=4.31 (t, 2H), δ=3.65 (m, 4H), δ=3.21 (t, 2H), δ=2.55 (m, 4H) ¹³C NMR (400 MHz, CDCl₃): 170.61, 169.28, 155.43, 134.96, 132.94, 129.71, 128.79, 129.43, 59.36, 57.95, 54.73, 52.71, 48.49, 48.04; HRMS(ESI) calcd. for C₁₅H₁₇ClN₄O₂S: 352.1 [M+H]⁺, found 352.7 [M+H]⁺.

### Preparation Example 8

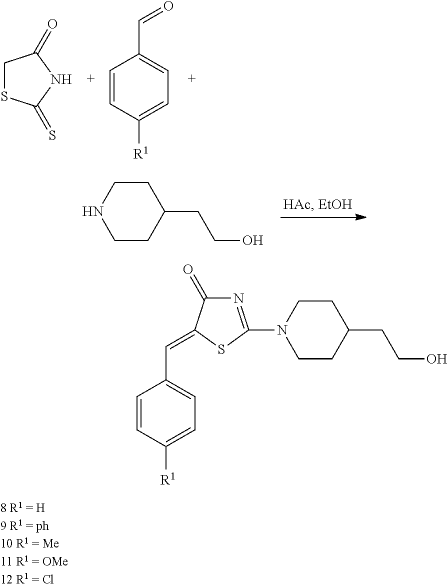

To a suspension of rhodanine (2.0 g, 15 mmol) in dry methanol (20 mL) and glacial acetic acid (0.1 mL), 4-(Hydroxyethyl) piperidine (2.9 g, 22.6 mmol) and benzaldehyde (22.6 mmol) under N₂, and the resulting mixture was further stirred under reflux for 2 h. Reaction was completed (checked by TLC analysis) and the obtained solution was cooled in an ice bath; the precipitated crystalline solid was filtered off, washed with cooled ethanol, and dried under vacuum to obtain the crude product. The crude product was subjected to silica gel column chromatography using dichloromethane/methanol (10:1) as mobile phase to afford the desired compound 8.

Yellow solid, Yield=46%. ¹H NMR (400 MHz, CDCl₃) δ=7.79 (s, 1H), δ=7.48 (d, 2H), δ=7.45 (m, 2H), δ=7.36 (t, 1H), δ=4.16 (t, 2H), δ=3.79 (m, 4H), δ=2.95 (m, 1H), δ=2.68 (t, 2H), δ=2.60 (m, 4H) HRMS(ESI) calcd. for C₁₇H₂₀N₂O₂S: 316.1 [M+H]⁺, found 316.7 [M+H]⁺.

### Preparation Example 9

Compound 9 was prepared with a method similar to preparation example 8, wherein benzaldehyde was replaced by 4-Biphenylcarboxaldehyde in this procedure.

Pale yellow solid, Yield=52.9%. ¹H NMR (400 MHz, CDCl₃) δ=7.90 (s, 1H), δ=7.63 (d, 2H), δ=7.57 (m, 4H), δ=7.38 (t, 2H), δ=7.30 (t, 1H), δ=5.54 (s, 1H), δ=4.27 (t, 2H), δ=3.63 (m, 4H), δ=2.88 (m, 1H), δ=2.69 (t, 2H), δ=2.17 (m, 4H) HRMS(ESI) calcd. for C₂₃H₂₄N₂O₂S 392.2 [M+H]⁺, found 392.5 [M+H]⁺.

### Preparation Example 10

Compound 10 was prepared with a method similar to preparation example 8, wherein benzaldehyde was replaced by p-methyl benzaldehyde in this procedure.

Pale yellow solid, Yield=49.1%. ¹H NMR (400 MHz, CDCl₃) δ=7.54 (s, 1H), δ=7.28 (d, 2H), δ=7.18 (t, 3H), δ=4.02 (t, 2H), δ=3.55 (m, 4H), δ=3.07 (m, 1H), δ=2.69 (t, 2H), δ=2.44 (m, 4H), δ=2.15 (s, 3H) HRMS(ESI) calcd. for C₁₈H₂₂N₂O₂S 330.1 [M+H]⁺, found 330.5 [M+H]⁺.

### Preparation Example 11

Compound 11 was prepared with a method similar to preparation example 8, wherein benzaldehyde was replaced by p-methoxybenzaldehyde in this procedure.

Pale yellow solid, Yield=47.6%. ¹H NMR (400 MHz, CDCl₃) δ=7.87 (s, 1H), δ=7.56 (d, 2H), δ=6.91 (d, 2H), δ=4.05 (t, 2H), δ=3.66 (s, 3H), δ=3.52 (m, 4H), δ=3.01 (m, 1H), δ=2.69 (t, 2H), δ=2.44 (m, 4H) HRMS(ESI) calcd. for C₁₈H₂₂N₂O₃S 347.1 [M+H]⁺, found 347.7 [M+H]⁺.

### Preparation Example 12

Compound 12 was prepared with a method similar to preparation example 8, wherein benzaldehyde was replaced by 4-Chlorobenzaldehyde in this procedure.

Pale green solid, Yield=37.2%. ¹H NMR (400 MHz, CDCl₃) δ=7.55 (s, 1H), δ=7.41 (dd, 4H), δ=4.87 (t, 2H), δ=3.98 (m, 4H), δ=2.65 (m, 1H), δ=2.27 (t, 2H), δ=2.05 (m, 4H) HRMS(ESI) calcd. for C₁₇H₁₉ClN₂O₂S 351.1 [M+H]⁺, found 351.6[M+H]⁺.

Experiment 1 the Inhibition Assay of Compounds 1˜12 to Mcl-1

FPA competition assays were adopted to evaluate the binding affinity of compounds to Mcl-1. The FITC labelled BH3 peptide of Mcl-1 (FITC-Mcl-BH3, FITC-AHx-KALETLRRVGD GVQRNHITAF-NH2) was synthesized and used as fluorescence probe in the FPA competition assays. The binding of FITC-Mcl-BH3 and Mcl-1 can enhance the polarized light. While the binding of compounds and Mcl-1 would reduce the polarization, IC₅₀ is the compound concentration that displaces 50% of the fluorescent ligand from the ligand-receptor complex.

All assays were performed in the assay buffer (pH 7.5) composed of 20 mM Tris, 50 mM NaCl, 3 mM DTT and 2% DMSO. The FPA competition assays were carried out in the 96-well, black, flat-bottom plates (Nunc). To check the interference effects of candidate compounds on the interaction between Mcl-1 and FITC-Mcl-BH3, solutions containing 250 nM Mcl-1 and 250 nM fluorescence probes were titrated with 250 μM selected compounds (0-128 μM, 2-times gradient) dissolved in the assay buffer. The emission fluorescence changes were recorded from 480 to 540 nm by TECAN Infinite M1000 Microplate Spectrophotometer with excitation wavelength being 450 nm. The IC₅₀ were calculated. The results are showed in table 1.

TABLE 1
The inhibitory effects of compounds 1~12 on Mcl-1.

| compound | IC₅₀ |
|---|---|
| 1 | 294.8 nM |
| 2 | 376.8 nM |
| 3 | 400.9 nM |
| 4 | 160.7 nM |
| 5 | 89.7 nM |
| 6 | 1.47 μM |
| 7 | 245.8 nM |
| 8 | 589.4 nM |
| 9 | 5.68 nM |
| 10 | 4.77 nM |
| 11 | 52.14 nM |
| 12 | 368.9 nM |

Experiment 2 Study of the Effects of Compounds 1˜12 on DLD-1 Cells Infected with pAd-PUMA.

The high-affinity interaction between Mcl-1 and compounds have been verified by above studies. The bioactivities of compounds to DLD-1 cells infected with pAd-PUMA were detected by Caspase-Glo® 3/7 Assay kit. DLD-1 cells suspension in logarithmic growth phase were cultured in 96-well, white, flat-bottom plates for 24 h before being infected by Ad-PUMA of suitable MOI. The infected DLD-1 cells were incubated for 24 h. Thereafter, 100 μl candidate compounds diluted in complete media (RPMI 1640 media supplemented with 10% FBS) were added to make the final compound concentrations range from 0 to 50 μM (2-times gradient), 10 points were selected for each compound. 24 h later, 25 μl apoptosis reagent from Caspase-Glo® 3/7 Assay kit was added in each tested well and incubate for 1 h. Shake gently and record the luminescence of each well on TECAN Infinite M1000 Microplate Spectrophotometer. The data were analyzed by Graphpad Prism. The results were detected by Caspase-Glo® 3/7 Assay kit following above descriptions. The results are showed in table 2.

TABLE 2
The effects of compounds 1~12 on DLD-1
cells infected with pAd-PUMA.

| Compound | IC₅₀ |
|---|---|
| 1 | 57.42 μM |
| 2 | 38.93 μM |
| 3 | 25.98 μM |
| 4 | 43.89 μM |
| 5 | 12.87 μM |
| 6 | 1.89 mM |
| 7 | 37.89 μM |
| 8 | 158.6 μM |
| 9 | 11.7 mM |
| 10 | 7.14 mM |
| 11 | 2.97 mM |
| 12 | 387.4 μM |

The above assays in PUMA-overexpressed cells and affinity activities showed apoptosis was significantly inhibited by compounds of the present invention, which suggests their high selectivity to target PUMA. These potential compounds can inhibit PUMA-dependent apoptosis, for treating radiation-induced hematopoietic damage, further enhance its prospect in the treatment of some diseases targeting PUMA overexpression, such as myocardial ischemia, ischemia-reperfusion injury, cardiac failure, Alzheimer's disease and AIDS virus infection as well.

1. A compound of formula I 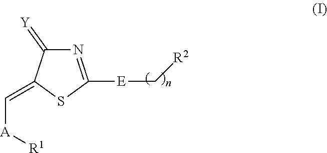
wherein,
A is C₅˜C₁₀ aryl, or 5˜10 membered heteroaryl containing 1˜3 atom(s) selected from oxygen, nitrogen or sulfur;
E is 5˜6 membered saturated or unsaturated aliphatic ring containing 0˜2 heteroatom(s) selected from oxygen, nitrogen or sulfur, or C₃˜C₆ alkyl, —NH—C₁˜C₃ alkyl, —O—C₁˜C₃ alkyl, —C₁˜C₃ alkyl-NH—C₁˜C₃ alkyl-, —C₁˜C₃ alkyl-O—C₁˜C₃ alkyl, —NH—C₁˜C₃ alkyl-NH—, —O—C₁˜C₃ alkyl-O—;
Y is O, S, NH or CH₂;
R¹ is H, C₁˜C₆ alkyl, C₁˜C₆ alkoxy, halogen, —OH, —NH₂, or 1˜3 R³-substituted C₅˜C₆ aryl, 5˜6-membered heteroaryl containing 1˜2 heteroatom(s) selected from oxygen, nitrogen, R³ is H, C₁˜C₆ alkyl, C₁˜C₆ alkoxy, halogen, —OH, —NH₂;
R² is H, —OH, —NH₂, —SH, —COOH, —CONH₂, —SO₂NH₂, —SO₃H, —CH(OH)—CH₂—OH;
n is integer from 1 to 5;
or a pharmaceutically acceptable salt thereof.

2. The compound of formula I or the pharmaceutically acceptable salt thereof according to claim 1,
wherein, Y is O.

3. The compound of formula I or the pharmaceutically acceptable salt thereof according to claim 1, wherein:
A is

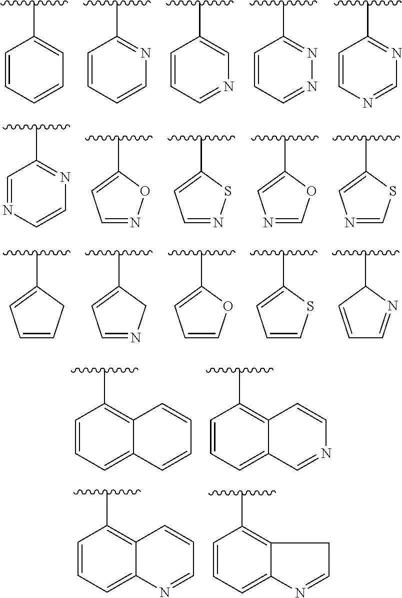

4. The compound of formula I or the pharmaceutically acceptable salt thereof according to claim 1, wherein:
A is C₅˜C₆ aryl, or 5˜6 membered heteroaryl containing 1˜2 heteroatom(s) selected from oxygen, nitrogen and sulfur;
E is 5˜6 membered saturated aliphatic ring containing 0˜2 heteroatom(s) selected from oxygen, nitrogen and sulfur.

5. The compound of formula I or the pharmaceutically acceptable salt thereof according to claim 1, wherein said compound or the pharmaceutically acceptable salt thereof is selected from compounds 1˜12 and their pharmaceutically acceptable salts: 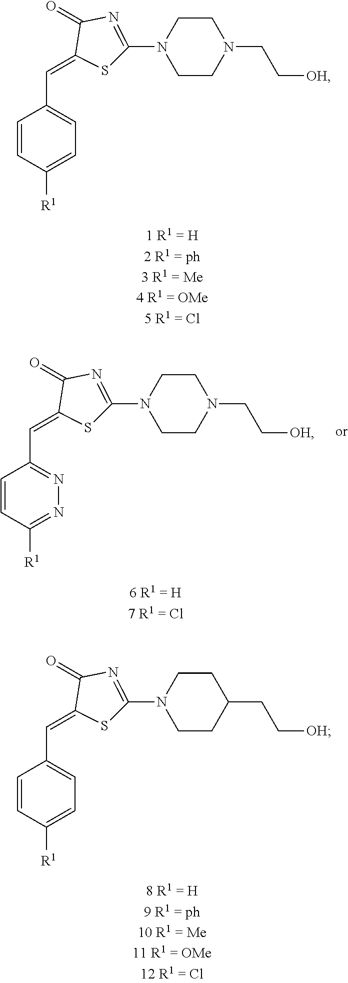
preferably, said compound or the pharmaceutically acceptable salt thereof is selected from the compounds as follows:

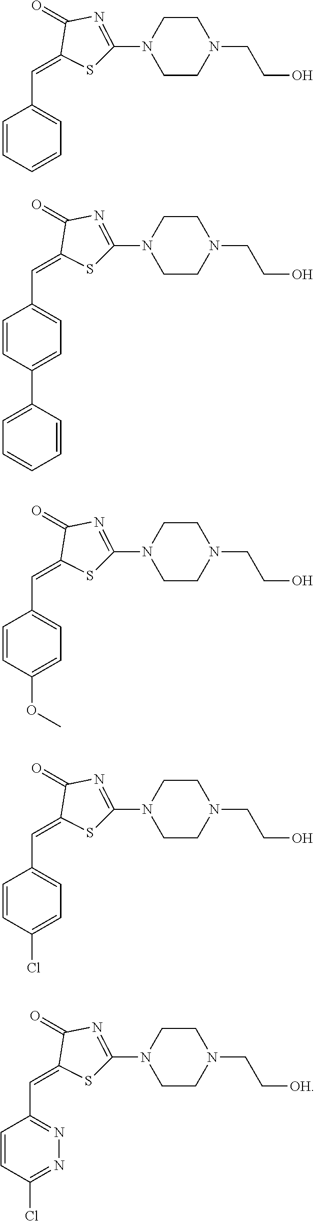

6. A method for preparing the compound of formula I or the pharmaceutically acceptable salt thereof according to claim 1, comprising the following steps: 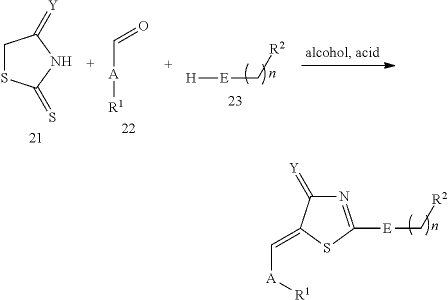
adding compounds 22 and 23 into the mixture of compound 21, alcohol and acid under N₂ atmosphere, the resulting mixture was further heated to reflux; when the reaction was completed, cooling the obtained solution, filtering off the precipitated crystalline solid, then washing and purifying by silica gel column chromatography;
wherein, A is C₅˜C₁₀ aryl, or 5˜10 membered heteroaryl containing 1˜3 atom(s) selected from oxygen, nitrogen or sulfur;
E is 5˜6 membered saturated or unsaturated aliphatic ring containing 0˜2 heteroatom(s) selected from oxygen, nitrogen or sulfur, or C₃˜C₆ alkyl, —NH—C₁˜C₃ alkyl, —O—C₁˜C₃ alkyl, —C₁˜C₃ alkyl-NH—C₁˜C₃ alkyl-, —C₁˜C₃ alkyl-O—C₁˜C₃ alkyl, —NH—C₁˜C₃ alkyl-NH—, —O—C₁˜C₃ alkyl-O—;
Y is O, S, NH or CH₂;
R¹ is H, C₁˜C₆ alkyl, C₁˜C₆ alkoxy, halogen, —OH, —NH₂, or 1˜3 R³-substituted C₅˜C₆ aryl, 5˜6-membered heteroaryl containing 1˜2 heteroatom(s) selected from oxygen, nitrogen, R³ is H, C₁˜C₆ alkyl, C₁˜C₆ alkoxy, halogen, —OH, —NH₂;
R² is H, —OH, —NH₂, —SH, —COOH, —CONH₂, —SO₂NH₂, —SO₃H, —CH(OH)—CH₂—OH;
n is integer from 1 to 5.

7. A method for treating a disease associated with PUMA protein comprising administering a therapeutically effective amount of the compound of formula I or the pharmaceutically acceptable salt thereof according to claim 1.

8. The method according to claim 7, wherein the disease is associated with radiation-induced damage or apoptosis.

9. The method according to claim 8, wherein said disease is radiation-induced hematopoietic damage, myocardial ischemia, Ischemia-reperfusion injury, cardiac failure, Alzheimer's disease or AIDS virus infection.

10. (canceled)

11. The compound of formula I or the pharmaceutically acceptable salt thereof according to claim 1, wherein,
E is:

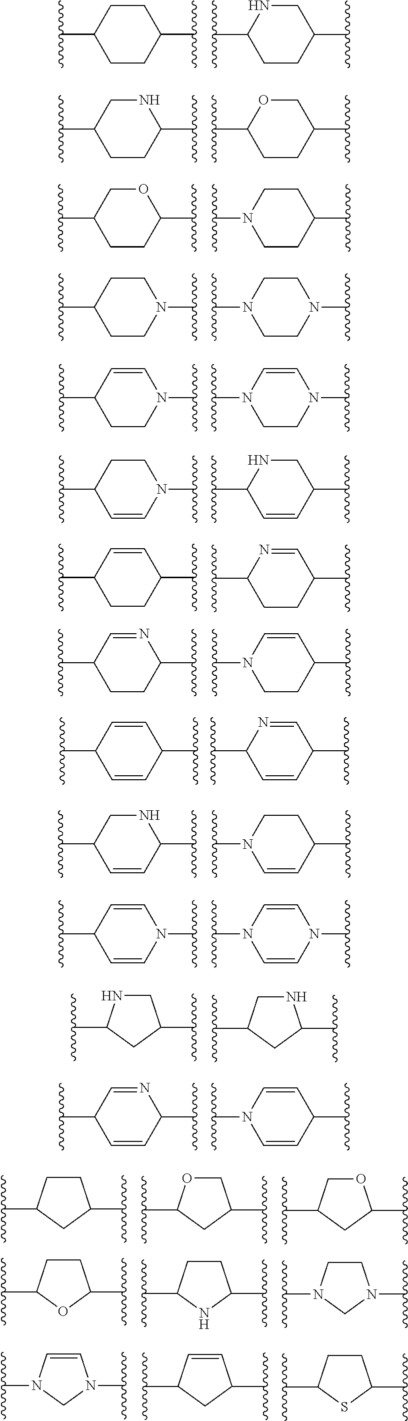

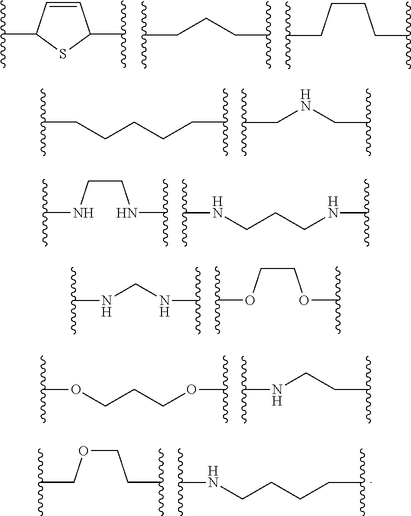

12. The compound of formula I or the pharmaceutically acceptable salt thereof according to claim 1, wherein,
R¹ is H, C₁˜C₆ alkyl, C₁˜C₆ alkoxy, halogen, —OH, —NH₂, or

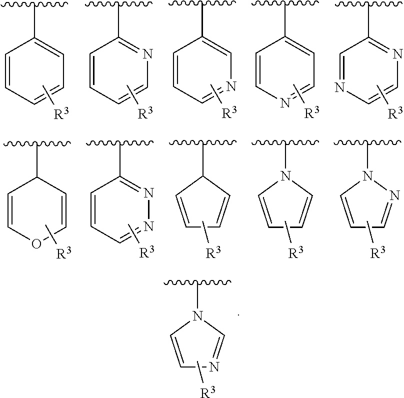

13. The compound of formula I or the pharmaceutically acceptable salt thereof according to claim 1, wherein, A is benzyl or pyridazinyl.

14. The compound of formula I or the pharmaceutically acceptable salt thereof according to claim 1, wherein, E is piperazinyl or piperidyl.
